(19) United States
(12) Patent Application Publication
(10) Pub. No.: US 2006/0200005 A1
(43) Pub. Date: Sep. 7, 2006

(54) Low profile, handle-in-between surgical scissors clamp

(75) Inventors: Todd M. Bjork, River Falls, MN (US); Todd William Sharratt, Stillwater, MN (US); Christopher Lee Berg, Anoka, MN (US)

Correspondence Address: SHEWCHUK IP SERVICES, 533 77TH STREET WEST, EAGAN, MN, 55121, US

(73) Assignee: LeVahn Intellectual Property Holding Company, LLC, St. Paul, MN (US)

(21) Appl. No.: 11/378,689

(22) Filed: Mar. 17, 2006

Related U.S. Application Data
(63) Continuation-in-part of application No. 10/664,195, filed on Sep. 17, 2003.
(63) Continuation-in-part of application No. 10/732,491, filed on Dec. 10, 2003.
Application No. 10/732,491 is a continuation of application No. 10/664,195, filed on Sep. 17, 2003.
(63) Continuation-in-part of application No. 11/034,231, filed on Jan. 12, 2005.
Application No. 11/034,231 is a continuation-in-part of application No. 10/732,491, filed on Dec. 10, 2003.
(60) Provisional application No. 60/535,910, filed on Jan. 12, 2004.

(51) Int. Cl. A61B 1/32

(52) U.S. Cl. 600/227

(57) ABSTRACT

A surgical clamp includes a first clamping member comprising a first clamping surface for engaging a first surgical rod and an attachment end. A second clamping member is mounted with respect to the first clamping member wherein the second clamping member comprises a second clamping surface for engaging a second surgical rod. A slide pin is disposed through and positioned within the attachment end of the first clamping member and wherein the shaft is in communication with the second clamping member. A force providing mechanism is disposed between the first and second clamping members and disposed about the slide pin wherein when the force providing mechanism and the slide pin are movable with respect to each other and place the first and second clamping members in either a loosened position or a tightened position.

### CROSS-REFERENCE TO RELATED APPLICATION(S)

This is a continuation-in-part of application Ser. No. 10/664,195 of Bjork et al. filed Sep. 17, 2003 and entitled FULCRUM WEDGE CLAMP, the content of which is hereby incorporated by reference in its entirety. This is also a continuation-in-part of application Ser. No. 10/732,491 of Bjork et al. filed on Dec. 10, 2003 and entitled FULCRUM WEDGE CLAMP, the content of which is hereby incorporated by reference in its entirety, which is a continuation of application Ser. No. 10/664,195. This application is also a continuation-in-part of application Ser. No. 11/034,231 of Bjork et al., filed Jan. 12, 2005 and entitled THREADED FULCRUM CLAMP, the contents of which is hereby incorporated by reference in its entirety, which is a continuation-in-part of application Ser. No. 10/732,491 and claims priority from Provisional Application No. 60/535,910 filed on Jan. 12, 2004.

### BACKGROUND OF THE INVENTION

The present invention relates generally to the field of surgical tools, and particularly to the design and manufacture of surgical retractor systems, including clamps for use in mounting surgical retractors with respect to an operating table. More particularly, the present invention relates to a clamp that is positioned into a tightened position with a wedge.

Surgical retractor systems are used during surgery to bias and hold tissue in a desired position. As one example, some surgical procedures require anterior access to the spine, through the patient's abdomen. Tissue such as skin, muscle, fatty tissue and interior organs needs to be held retracted to the side so the surgeon can obtain better access to the vertebrae structures of primary interest.

Surgical retraction may be performed by one or more aides using handheld tools, with the most basic retractor apparatus being a tongue depressor. More commonly now in sophisticated operating rooms during abdominal or chest surgery, a retractor support apparatus is typically disposed about the surgical site. The retractor assembly may, for instance, include a ring or support frame which is rigidly supported from the patient's bed above and around the surgical incision location, with a number of clamps and retractor blades to hold back tissue proximate to the surgical incision. Other retraction systems, such as those disclosed in U.S. Pat. Nos. 6,315,718, 6,368,271 and 6,659,944 to Sharratt, incorporated herein by reference, may not include a ring and/or may be directed at other types of surgery. Retractor clamps are also commonly used to mount the retractor support apparatus with respect to an operating table, support post and/or part of the bed frame. Some retractor clamps are manufactured in a captivated configuration so that users cannot disassemble the retractor clamp.

A retractor clamp typically includes a first clamping member, a second clamping member and a handle. In one style of clamping member, the clamping member is fabricated from a unitary structure that is generally in the shape of the letter “U”. In each of the retractor clamps, the object to be clamped is placed between the legs of the U-shaped structure so that the object is proximate the base of the U-shaped structure. Movement of the legs of the U-shaped structure towards each other causes the object to be clamped with respect to the clamping member.

The use of prior art clamping members having U-shaped structures has some disadvantages in particular surgical procedures. First, the U-shaped structure must be disposed over an end of a retractor support frame or ring apparatus and slid longitudinally into a desired position. When previously placed retractor clamps are disposed between the end of the support arm and the desired location, the interfering clamps must be removed to allow the additional surgical clamp to be disposed in the desired location. Having to disassemble at least a portion of the surgical support apparatus about a surgical site to add additional retractors adds unnecessary additional time and expense to the surgical procedure.

Additionally, the prior art clamps may position one of the U-shaped structures and the handle above the first U-shaped structure and the retractor support frame. Because the second U-shaped structure and the handle are located above the surgical support frame, the second U-shaped structure and the handle may obstruct access to the surgical site during some surgical procedures.

In devising a proper clamping structure, the clamp should give the surgeon flexibility in quickly assembling the retraction system and in placement of the various retractors. Once the various retractors are in place and oriented and pulled as desired, the retraction system clamps should allow quick and easy tightening so the entire retraction system is maintained fixedly in place. Once tightened the retraction system should be unobtrusive so neither the tissue held retracted nor the retraction system interfere in any way with the surgeon or the surgical procedure. After surgery is completed (or perhaps once or more during surgery), the retraction system should quickly loosen and/or disassemble so as relax the retracted tissue and minimize damage to the retracted tissue. Surgical retractor systems must be robust and strong, as even a slight possibility of failure during use is not tolerated. Surgical retractor assemblies should be readily reusable, including sterilizable, for use in multiple surgeries.

Surgical retractor systems should maintain a relatively low cost. Improvements in surgical retractor clamps and systems can be made in keeping with these goals.

### BRIEF SUMMARY OF THE INVENTION

The present invention is a clamp and a support system using the clamp. The clamp has a first clamping opening for receiving the support frame rod and second clamp opening for receiving the retractor or tool shaft. A handle controls operation of at least one and preferably both of the clamp members. In one aspect, the handle is always in line with one of the support frame rod and the retractor shaft, enabling tightening with a single handed scissors action. In another aspect, the handle is attached to the clamp at a location between the first and second clamping openings. In another aspect, the clamp is very low profile and positioned for minimal interference with the surgical arena, providing minimal underclearance for the support frame rod, and minimal overclearance for the retractor shaft, and minimal height separation between the support frame rod and the retractor shaft. In another aspect, the handle can be moved from a loosened position to a tightened position with a balanced, single hand application, enabled in part by use of a clamping force provided by a slide pin.

### BRIEF DESCRIPTION OF THE DRAWINGS

FIG. 1 is a perspective view of the surgical clamp of the present invention in a loosened position.

FIG. 2 is a perspective view of the surgical clamp of the present invention in a tightened position.

FIG. 3 is an exploded view of the surgical clamp of the present invention.

FIG. 4 is a perspective view of an actuating mechanism of the surgical clamp of the present invention.

FIG. 5 is a top view of the actuating mechanism of the surgical clamp of the present invention.

FIG. 6 is a partially exploded perspective view of the first alternative embodiment of the surgical clamp of the present invention.

FIG. 7 is a side view of the first alternative embodiment of the surgical clamp of the present invention.

FIG. 8 is a sectional view of the second alternative embodiment of the surgical clamp of the present invention.

FIG. 9 is an exploded view of the second alternative embodiment of the surgical clamp of the present invention.

FIG. 10 is a perspective view showing the outline of the clamp of FIG. 1 in dashed lines to explain the relationship between the attachment locations of the clamp.

FIG. 11 is a cross-sectional side view showing the mapping of FIG. 10 with the clamp oriented with both the retractor support arm and the retractor handle shaft extending horizontally.

FIG. 12 is a cross-sectional side view showing the clamp of FIG. 7 in dashed lines to explain the relationship between the attachment locations of the clamp.

FIG. 13 is a cross-sectional side view similar to FIG. 12 but with the retractor handle shaft pivoted 90° relative to the retractor support arm and the cut plane moved forward correspondingly with the midpoint connection line.

FIG. 14 is a cross-sectional side view similar to FIG. 12 but with the retractor handle shaft pivoted 180° relative to the retractor support arm.

FIG. 15 is the cross-sectional side view of FIG. 8 mapped to explain the relationship between the attachment locations of the clamp.

While the above-identified drawing figures set forth preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

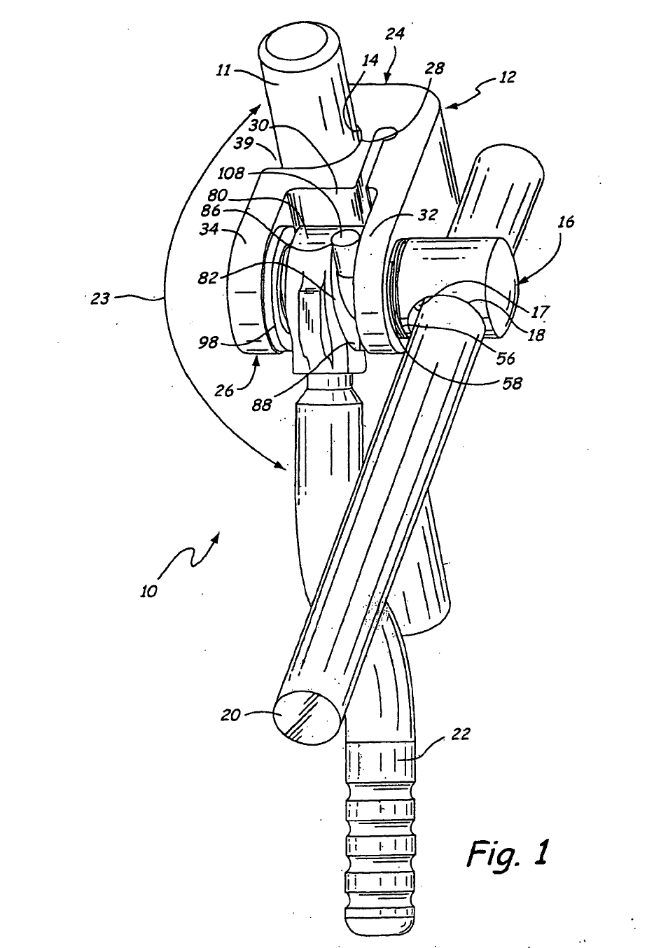

The present invention includes a low profile surgical clamp generally illustrated in FIG. 1 at 10. The clamp 10 is particularly suited for mounting a retractor (not shown) with respect to an operating table (not shown). The clamp 10 includes a first clamping member 12 having a first clamping surface 14 and a second clamping member 16 having a second clamping surface 18. In some aspects of the invention, other components may be substituted for the second clamping member 16 such as, but not limited to, permanently attached retractors, or retractors that are attached using other types of clamps or fasteners.

To position the clamp 10 within the surgical site, a clamping end 24 of the first clamping member 12 is positioned proximate a bar of a retractor support system, such as a retractor support arm 11. The first clamping surface 14 proximate the clamping end 24 is disposed about a portion of the retractor support arm 11.

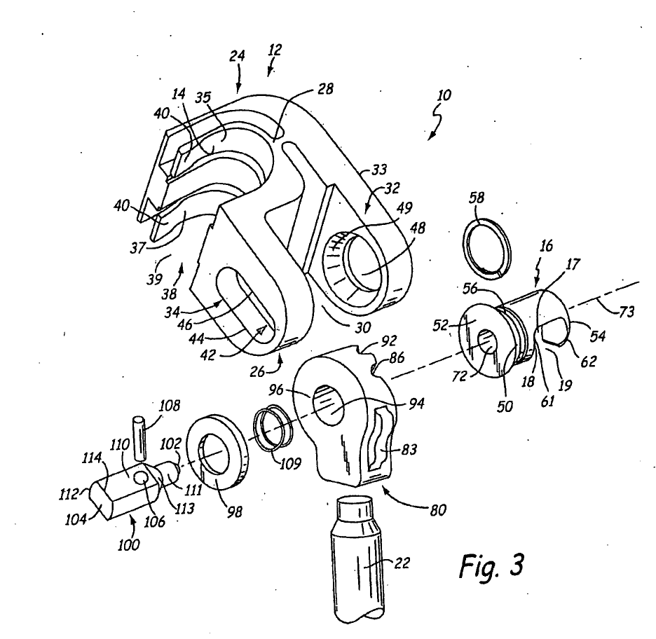

The first clamping member 12 also includes an attachment end 26 and a fulcrum portion 28 between the clamping end 24 and the attachment end 26. The first clamping member 12 has a recess 30 defined by an upper leg portion 32 and a lower leg portion 34 proximate the attachment end 26. The fulcrum portion 28 extends between the upper leg portion 32 and the lower leg portion 34 as best illustrated in FIG. 3. The fulcrum portion 28 flexes to allow the upper leg portion 32 to move with respect to the lower leg portion 34 so that the retractor support arm 11 may be clamped within the first clamping surface 14.

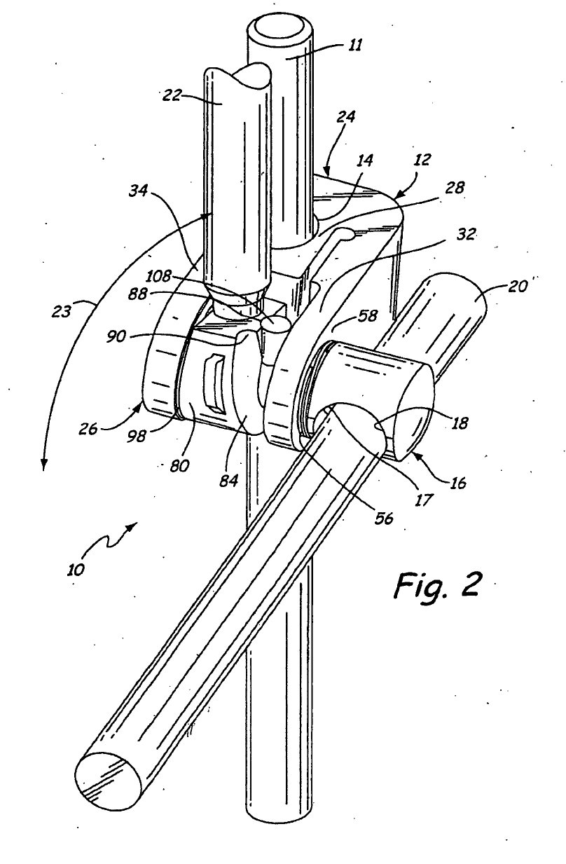

An actuating mechanism 80 is disposed within the recess 30 between the upper leg portion 32 and the lower leg portion 34 of the first clamping member 12 as illustrated in FIGS. 1 and 2. The actuating mechanism 80 preferably is a wedge. By the term wedge is meant a component that progressively increases in cross-sectional thickness such that when the wedge is moved or rotated in a restricted space, the movement or rotation of the wedge causes progressive increase in thickness to cause a force to be applied, such as a leverage action, to spread apart leg portions of the clamp. The wedge may take on forms other than those specifically described herein.

The wedge 80 is movable within the recess 30 and forces the upper and lower leg portions 32, 34 apart and causes the fulcrum portion 28 to flex. As the fulcrum portion 18 flexes, the first clamping member 12 constricts and the opening defined by the first clamping surface 14 changes dimensions to tighten about the rod of the retractor support arm 11. When constricted, the first clamping surface 14 frictionally engages the retractor support arm 11 in a tightened relationship.

In a preferred embodiment shown in FIGS. 1-5, the first clamping surface 14 is configured to generally conform to the cylindrical shape (circular cross-sectional configuration) of the retractor support arm 11. The first clamping surface 14 may alternatively have a different shape, and thereby be configured to conform to rods of different retractor support arms with other shapes and cross-sections.

Referring to FIG. 3, the first clamping surface 14 defines a clamping slot 38 that is open for transverse attachment onto the retractor support arm 11, i.e., that permits the first clamping member 12 to be placed on the retractor support arm 11 without threading the retractor support arm 11 through the clamp and without moving any other surgical equipment that has been previously disposed upon the retractor support arm 11. Further, in the usual orientation of the clamp 10, the clamping slot 38 is open from below. The clamping slot 38 therefore permits the first clamping member 12 to be placed from above onto the underlying retractor support arm 11 and pressed downward into loosened attachment on the retractor support arm 11.

When the clamp 10 is in a loosened position, the first clamping member 12 is retained upon the retractor support arm 11 by a constricted entrance 39 to the clamping slot 38. In an exemplary embodiment, a bead or raised surface 40 is disposed along a length of resilient portions 35, 37 that move as the retractor support arm 11 is positioned within the clamping slot 38 where the bead 40 constricts the entrance 39. However other devices that constrict the entrance 39 to the clamping slot 38 are within the scope of the present invention including, but not limited to, a clip, a roller or a spring loaded device.

The constricted entrance 39 provides a preliminary clamping force around the portion of the retractor support arm 11 such that the constricted entrance 39 to the clamping slot 38 prevents the clamp 10 from slipping off the retractor support arm 11 when the clamp 10 is in loosened position. The entrance 39 of the clamping slot 38 should not be so constricted to prevent use of manual force to position the first clamping member 12 about the portion of the retractor support arm 11. However, the entrance 39 of the clamping slot 38 should be sufficiently constricted to prevent the first clamping member 12 from accidentally slipping off of the retractor support arm 11. An exemplary amount of constriction of the entrance 39 of the clamping slot 38 is between about 0.010 inches and 0.020 inches and preferably about 0.015 inches.

The second clamping member 16 extends through a through bore 48 defined by a frustro-conical surface 49 within the upper leg portion 32 of the first clamping member 12.

The second clamping member 16 includes a frustro-conical surface 50 at a proximal end 52 that cooperates with the frustro-conical surface 49.

With the second clamping member 16 being positioned within the upper leg portion 32, an annular groove 56 on the second clamping member 16 is positioned above an upper surface 33 of the upper leg portion 32. A snap ring 58 is disposed within the annular groove 56 and rotatably captivates the second clamping member 16 within the upper leg portion 32 of the first clamping member 12 as illustrated in FIGS. 1 and 2.

Referring to FIGS. 1-3, the second clamping surface 18 is positioned on a distal end portion 54 of the second clamping member 16. A retractor handle shaft 20 is disposed within an opening 17 defined by the second clamping surface 18. An arcuate upper portion 62 and an end 102 of the slide pin 100 constrict an entrance 19 of the opening 17 and retain the retractor shaft 20 within the second clamping surface 18. Although the second clamping member 16 is described and illustrated in the drawings as retaining and clamping a retractor handle shaft 20, the second clamping member 16 can also retain and clamp other surgical devices. By surgical devices is meant any element that is useful in conducting a surgical procedure including, but not limited to, a retractor support apparatus, a retractor or any other medical instrument that is used during a surgical procedure such as a camera, a light or a catheter.

The second clamping member 16 is positioned into a tightened position at approximately the same time that the wedge 80 forces the first and second leg portions 32, 34 apart. When the second clamping member 16 is in the tightened position, the second clamping member 16 is non-rotatably fixed with respect to the first clamping member 12 and the retractor handle shaft 20 is frictionally engaged within the second clamping surface 18.

In the tightened position, the second clamping member 16 is rotatably fixed within the upper leg portion 32 by a frictional engagement of the cooperating frustro-conical surfaces 49, 50. The retractor handle shaft 20 is frictionally engaged between the arcuate upper portion 62 and an end 102 of a slide pin 100 positioned through a through bore 72. The through bore 72 extends through the second clamping member 16 along an axis 73 and intersects a bottom portion 61 of the clamping surface 18.

Referring to FIG. 3, an upper substantially circular cross-section portion 111 of the slide pin 100 extends through an elongated slot 42 within the lower leg portion 34, a washer 98, a compression spring 109, a through bore 94 of the wedge 80, the through bore 48 of the upper leg portion 32 and into the through bore 72 of the second clamping member 16. The end 102 of the upper portion 111 extends into the opening 17.

A lower portion 110 of the slide pin 100 captivates the washer 98 and the compression spring 109 between the lower leg portion 34 and the wedge 80. With the slide pin 100 disposed within the clamp 10, a shoulder 113 is positioned proximate the proximal end 52 of the second clamping member 16. The shoulder 113 cooperates with the proximal end 52 to prevent the slide pin 100 from being further inserted into the clamp 10.

Referring to FIGS. 1-3, the washer 98 is positioned between a bottom surface 96 of the wedge 80 and the lower leg portion 34. The washer 98 prevents wear of the lower leg portion 34 and the wedge 80 as the clamp 10 is used over time.

The lower portion 110 of the slide pin 100 has substantially flat, parallel sides 112, 114 that cooperate with substantially flat, parallel sides 44, 46, respectively, of the elongated slot 42. The cooperation of the lower portion 110 within the elongated slot 42 prevents rotation of the slide pin 100 within the clamp 10 while allowing the slide pin 100 to move slightly longitudinally with respect to the first and second clamping members 12, 16.

The slide pin 100 includes a bore 106 that is in a substantially perpendicular relationship with the axis 73. With the lower portion 110 of the slide pin 100 positioned within the elongated slot 42, the bore 106 is positioned between the proximal end 52 of the second clamping member 16 and the wedge 80. A height pin 108 is positioned through the bore 106 in the slide pin 100.

The compression spring 109 biases the wedge 80 toward the second clamping member 16 such that first and second ramped surfaces 82, 84 of the wedge 80 engage the height pin 108. The engagement of the height pin 108 with the first and second ramped surfaces 82, 84 of the wedge 80 prevents the slide pin 100 from exiting from the lower leg portion 34. The height pin 108 also holds the end 102 of the slide pin 100 into the opening 17 and presses the shoulder 113 into the proximal end 52 of the second clamping member 16.

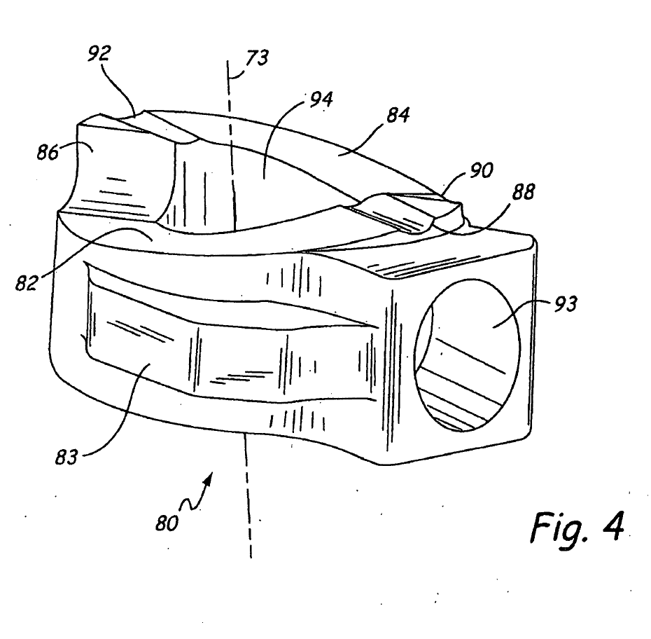

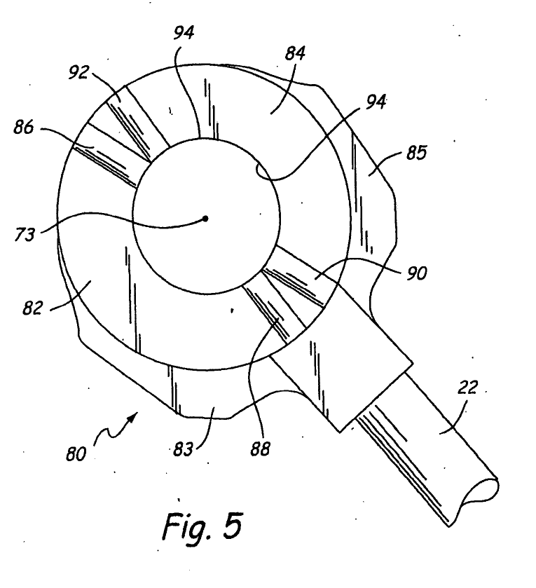

Referring to FIGS. 4 and 5, the wedge 80 includes the first and second ramped surfaces 82, 84 that are symmetric about the axis 73. Arcuate base indentions 86, 90 are located each at a base of the first and second ramped surfaces 82, 84, respectively, and opposite each other. Arcuate apex indentions 88, 92 are located each at an apex of the first and second ramped surfaces 82, 84, respectively, and opposite each other.

The height pin 108 engages the base indentions 86, 90 each at the base of the first and second ramped surfaces 82, 84 to retain the wedge 80 in a loosened position where the first and second clamping members 12, 16, are sized to loosely receive the support arm 11 and the retractor handle shaft 20, respectively. The height pin 108 engages the apex indentions 88, 92 to retain the wedge 80 in a tightened position where the first and second clamping members 12, 16, have slightly smaller opening dimensions to frictionally engage the support arm 11 and the retractor handle shaft 20, respectively.

In operation, the wedge 80 is in the loosened position when the height pin 108 is engaged with the base indentions 86, 90 as best illustrated in FIG. 1. With the wedge 80 in the loosened position, the first clamping member 12 and the second clamping member 16 can accept the retractor support arm 11 and the retractor handle shaft 20, respectively, and the second clamping member 16 is rotatable with respect to the first clamping member 12.

The first clamping member 12 is disposed in a selected position on the retractor support arm 11 by positioning the constricted entrance 39 of the clamping slot 38 proximate the retractor support arm 11. Manual force is transversely applied to the first clamping member 12 substantially perpendicular to an axis of the retractor support arm 11 to overcome the constricted entrance 39 of the clamping slot 38, thereby disposing the first clamping member 12 about the retractor support arm 11. With the wedge 80 in the loosened position, the first clamping member 12 is slidably positionable on the retractor support arm 11. In the loosened position, the first clamping member 12 also permits substantially free rotation of the clamp 10 about the axis of the retractor support arm 11.

With the first clamping member 12 positioned on the retractor support arm 11, the retractor handle shaft 20 is positioned proximate the constricted entrance 19 of the opening 17 within the second clamping member 16. Manual force is applied substantially perpendicular to an axis of the retractor handle shaft 20 to overcome the bias of the compression spring 109 and force the end 102 of the slide pin 100 from the opening 17 and dispose the retractor handle shaft 20 within the opening 17. With the retractor handle shaft 20 positioned within the opening 17, the end 102 of the slide pin 100 is biased back into the opening 17 such that the retractor handle shaft 20 is slidably retained within the second clamping member 16. While slidably retained within the second clamping member 16, the retractor handle shaft 20 can also be rotated about its longitudinal axis, so the surgeon can select the most desired orientation of the retractor blade (not shown).

A significant advantage of the second clamping member 16 is that it includes a clamping opening 17 which is open from above the retractor support arm 11. The clamping opening 17 therefore permits the retractor handle shaft 20 to be transversely placed into the second clamping member 16 after the first clamping member 12 has been positioned on the retractor support arm 11 and without requiring threading of the retractor handle shaft 20 through the clamping opening 17.

To position the wedge 80 into the tightened position, a handle 22 fixedly attached to the wedge 80 is moved in the direction of arrows 23 as illustrated in FIG. 1, which rotates the wedge 80 about the slide pin 100 and axis 73. The handle 22 provides a mechanical advantage in moving the wedge 80. Although the preferred embodiment includes a rotatable wedge, one skilled in the art will recognize that moving a wedge in other manners such as sliding is also within the scope of the invention. The purpose of the wedge 80 is to increase the wedge's thickness within the recess 30 to force the upper and lower leg portions 32, 34 apart.

As the wedge 80 is rotated about the slide pin 100, the height pin 108 moves along the first and second ramped surfaces 82, 84 toward the apexes of the first and second ramped surfaces 82, 84. As the height pin 108 rises on the first and second ramped surfaces, 82, 84, an increasing force is placed upon the height pin 108. The wedge 80 includes first and second strengthening portions 83, 85 to stiffen the wedge 80 and prevent the wedge 80 from flexing as the wedge 80 is rotated, as best illustrated in FIGS. 1-5. A maximum force is placed upon the height pin 108 when the wedge 80 is positioned into the tightened position just before the height pin 108 is disposed within the apex indentions 88, 92 as best illustrated in FIG. 2. An advantage of using the wedge 80 is that the wedge 80 places equal and opposite forces on the upper and lower leg portions 32, 34. Once the first clamp 12 is closed to a tightened position, it does not require further application of force or holding by the surgical staff to remain in the tightened position.

Referring to FIGS. 1 and 2, as the wedge 80 is rotated from the loosened position to the tightened position, the wedge 80 first compresses and bottoms out the compression spring 109. As the wedge 80 is further rotated from the loosened position to the tightened position, the wedge 80 forces the first and second leg portions 32, 34 apart by forcing the shoulder 113 of the slide pin 100 into the proximal end 52 of the second clamping member 16 and transferring the force to the first leg portion 32. With the first and second leg portions 32, 34 forced apart, the fulcrum portion 28 flexes which causes the first clamping slot 38 to constrict such that the first clamping surface 14 frictionally engages the retractor support arm 11.

With the wedge 80 in the tightened position, the shoulder 113 is forced upward and into the proximal end 52 of the second clamping member 16 and creates a frictional engagement between the cooperating frustro-conical surface 49 of the through bore 48 in the upper leg portion 32 and the frustro-conical surface 50 of the second clamping member 16. The frictional engagement of the frustro-conical surfaces 49, 50 prevents rotational movement of the second clamping member 16 with respect to the first clamping member 12.

With the wedge 80 in the tightened position, the end 102 of the slide pin 100 is raised into the opening 17 through the through bore 72. The raised end 102 of the slide pin 100 contacts the retractor handle shaft 20 and creates a frictional engagement between the arcuate upper portion 62 of the second clamping surface 18, the retractor handle shaft 20 and the end 102 of the slide pin 100.

One skilled in the art will recognize that a plane of movement of the handle 22 approximately intersects an axis of the retractor support arm 11 as best illustrated in FIGS. 1 and 2. Therefore, the operator can conveniently manipulate the wedge 80 from the loosened position to the tightened position through a scissors action, by gripping the handle 22 and the retractor support arm 11 with one hand and forcing the handle 22 toward the retractor support arm 11. The tightened orientation of the handle 22 has the longitudinal axis of the handle 22 extending generally parallel to the retractor support arm 11 to effectuate the scissors tightening action. In the most common orientation of use of the clamp 10, the support arm 11 and the retractor handle shaft 20 will both extend horizontally, such that the handle 22 pivots in a horizontal handle plane. Regardless of orientation, the handle plane always substantially contains or is parallel to the axis of the retractor support arm 11, so the retractor support arm 11 can be used in the single-handed squeezing of the scissors action.

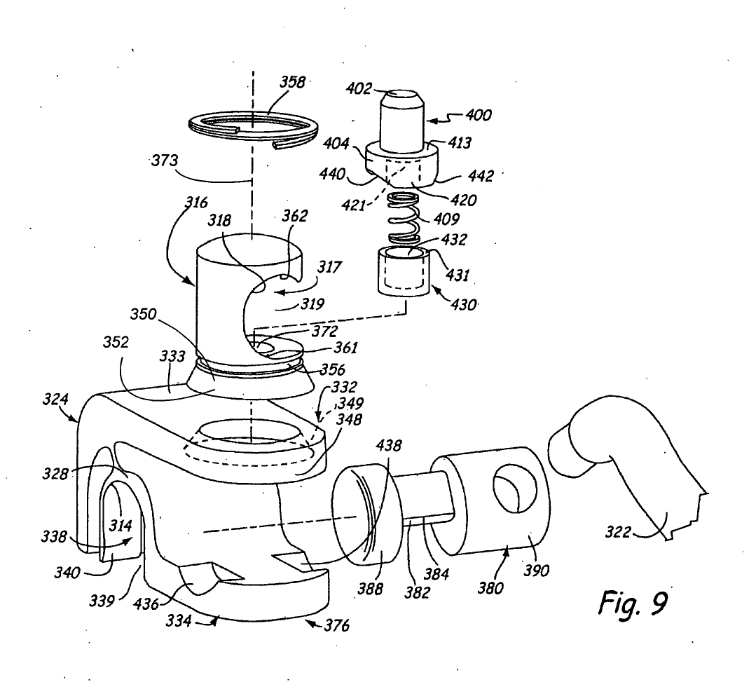

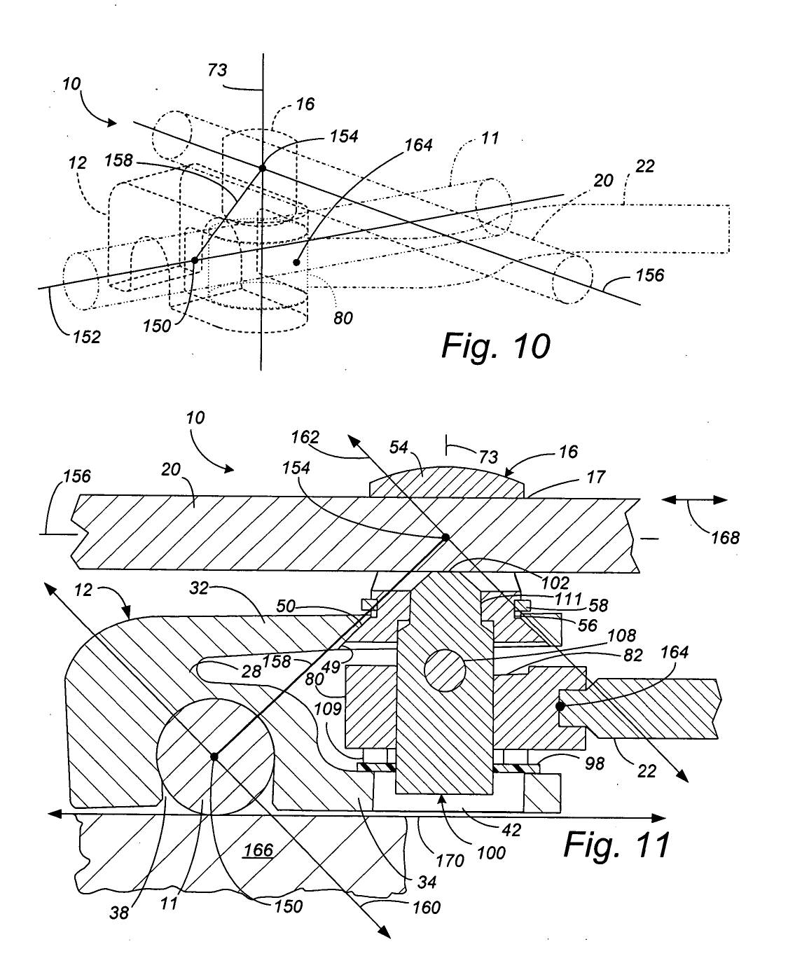

The surgical clamp 10 of the present invention also provides a low profile clamping device for conducting the surgical procedure. What is meant by low profile is that the handle 22 is disposed proximate the retractor support arm 11 and the retractor handle shaft 20 as best illustrated in FIGS. 1 and 2 and further described below with reference to FIGS. 10-15. By disposing the handle 22 in a low profile position and proximate the retractor support arm 11 and proximate the retractor handle shaft 20, the surgical site remains relatively open and free of obstruction thereby providing better access to the surgical site.

Another advantage is achieved by having the second clamp member 16 and the handle 22 both on the same side of the first clamp member 12; in the most common orientation of the clamp 10, both the handle 22 and the second clamp member are outside the first clamp member 12 relative to the surgical arena rather than having one inside and the other outside the first clamp member. The combined features of having the second clamp member 16 and the handle 22 both on the same side of the first clamp member 12 in combination with scissors operation of the handle 22 with the bar 11 received in the first clamp member 12 can only be achieved by a design which places the handle 22 between the two clamp members 12, 16, a design which is very compact, or (as in this embodiment) a design which is both “handle-in-between” and compact.

An advantage of the clamp 10 of the present invention is that the clamp 10 does not have to be slid along the retractor support arm 11 to a new selected position. The retractor clamp 10 can be repositioned on the support arm 11 by first positioning the wedge 80 into the loosened position such that the first clamping slot 38 is not constricted and detaching the first clamping member 12 from the retractor support arm 11. The first clamping member 12 is detachable from the retractor support arm 11 by applying manual force in an opposite direction as used to position the first clamping member 12 on the support arm 11. After the clamp 10 has been removed from the retractor support arm 11, the clamp 10 is repositionable on the retractor support arm 11 by positioning an entrance 39 to the clamping slot 38 against the retractor support arm 11 and applying manual force substantially perpendicular to the axis of that portion of the retractor support arm 11.

The retractor handle shaft 20 can also be repositioned within the second clamping member 16 without having to slide the retractor handle shaft 20 with respect to the second clamping surface 18. The retractor handle shaft 20 is removed from the second clamping member 16 by providing manual force in the opposite direction of the force used to position the retractor handle shaft 20 within the opening 17 of the second clamping member 16. The retractor handle shaft 20 can be reinserted into the opening 17 by reapplying manual force generally perpendicular to the axis of the retractor handle shaft 20.

The surgical clamp 10 of the present invention, having the first and second clamping surfaces 14, 18, enables the clamp 10 to be positioned upon the retractor support arm 11 in a selected position. Further, an additional retractor can be easily and conveniently positioned within a surgical site without having to thread the end of the retractor handle shaft through the opening 17 defined by the second clamping surface 18. The convenience of the surgical clamp 10 of the present invention allows the surgical site to be quickly assembled, modified during a surgical procedure, and disassembled, which enables a surgical team to conduct a more efficient surgical procedure.

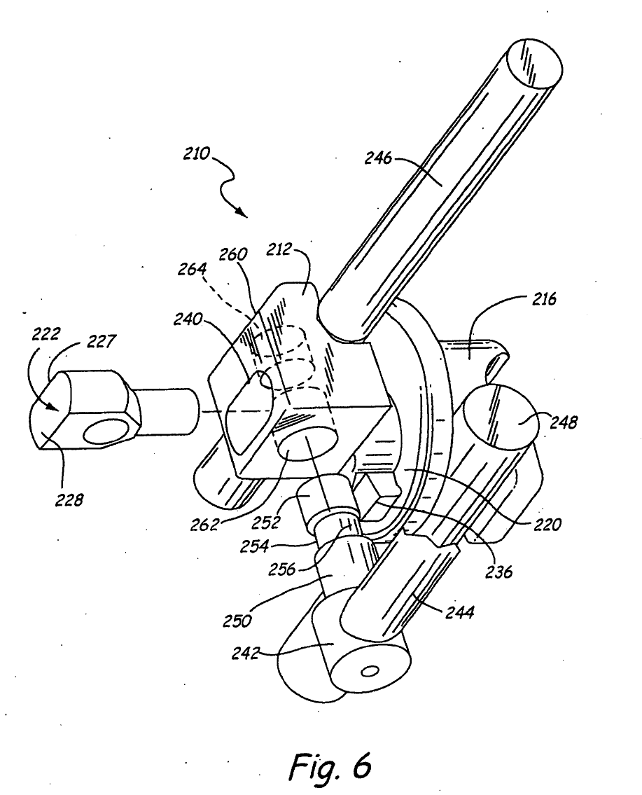

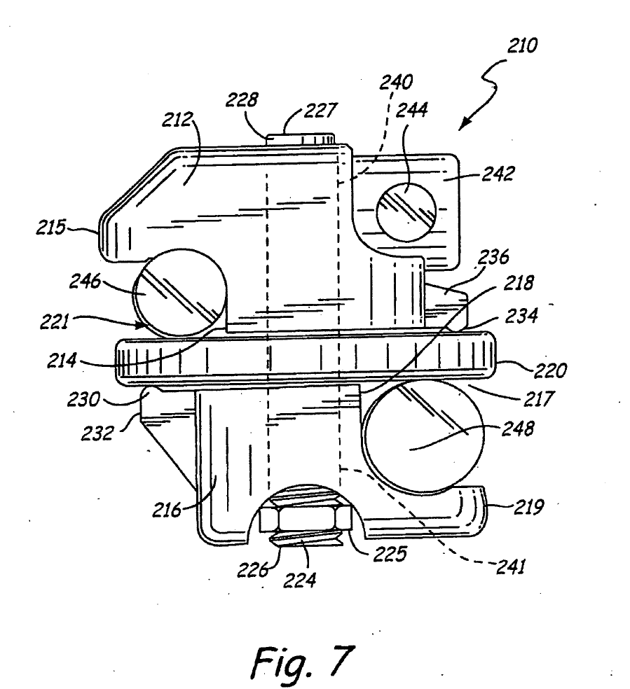

An alternative embodiment of the surgical clamp of the present invention is illustrated in FIGS. 6 and 7 at 210. The clamp 210 includes a first clamping member 212 and a second clamping member 216 separated by a washer 220. A tension bolt 222 is disposed through the first and second clamping members 212, 216. The tension bolt 222 includes an externally threaded portion 224 proximate a first end 226, a head 228 proximate a second end 227 and an intermediate portion (not shown) between the head 228 and the threaded portion 224.

The threaded portion 224 of the tension bolt 222 is disposed through an elongated bore 240 within the first clamping member 212 and through a bore 241 within the second clamping member 216. A nut 225 threadably engages the threaded portion 224 and retains the first and second clamping members 212, 216 about the tension bolt 222.

The first clamping member 212 is preferably made of a unitary structure having a surface 214 defining a first clamping slot 221 proximate a first end 215 and a first pivot surface 234 proximate a second end 236 which contacts an upper surface of the washer 220. The first clamping member 212 includes the elongated bore 240 for accepting the head 228 of the tension bolt 222.

The first clamping member 212 includes an integral collar 260 that cooperates with a camming pin 242. The camming pin 242 includes end portions 250, 252 and an intermediate portion 254. The end portions 250, 252 and the intermediate portion 254 are generally cylindrical in shape and are located adjacent one to another. The end portions 250, 252 are centered about a rotational axis and are captivated within first and second through bores 262, 264. The captivated end portions 250, 252 rotatably support the intermediate portion 254 within the collar 260 and a through bore within the head 228 of the tension bolt 222.

The tension bolt 222 is positioned through the elongated bore 240 within the first clamping member 212 including the integral collar 260 and the through bore 241 within the second clamping member 216, such that the head 228 is disposed within the elongated bore 240. The elongated bore 240 is elongated to allow the required movement of a head 228 of the tension bolt 222 when the clamp 210 is positioned from the loosened position to the tightened position and also in the reverse direction. The head 228 includes the through bore that is aligned with the first and second through bores 262, 264 within the collar 260 and engages the intermediate portion 254 of the camming pin 242.

The intermediate portion 254 is eccentrically coupled between the end portions 250, 252. The intermediate portion 254 includes an outer circumferential surface 256 having an axis that is spaced from the rotational axis the camming pin 242 by a selected distance. The distance separating the axis of the camming pin 242 and the axis of the intermediate portion 256 generally determines the maximum distance that the camming pin 242 moves the tension bolt 222 relative to the first and second clamping members 212, 216. Preferably, the distance separating the axis of the camming pin 242 and the axis of the intermediate portion 254 is sufficient to frictionally secure first and second surgical rods 246, 248 within the first and second clamping members 212, 216, respectively.

The second clamping member 216 is also preferably a unitary structure having a surface 218 defining a second clamping slot 217 proximate a first end 219 and a second pivot surface 230 proximate a second end 232. The second pivot surface 230 contacts a bottom surface of the washer 220.

In operation, a handle 244, fixedly attached to the camming pin 242, is in a loosened position such that the first and second clamping slots 221, 217 of the first and second clamping members 212, 216 accept first and second support members 246, 248, respectively. The first support member 246 can be, for instance, a handle shaft of a retractor blade, and the second support member 248 can be, for instance, a retractor support frame. The movement of the handle 244 from the loosened position to a tightened position causes the intermediate portion 254 to engage the tension bolt 222. As the tension bolt 222 is engaged, a force is applied to the first clamping member 212.

The force causes the first clamping member 212 to pivot about the first pivot surface 234 such that the first support member 246 is frictionally engaged between the first clamping member 212 and the upper surface of the washer 220. The force is also applied through the tension bolt 222 to the second clamping member 216 and causes the second clamping member 216 to pivot about the second pivot surface 230 and causes the second support member 248 to be frictionally engaged between the bottom surface of the washer 220 and the second clamping member 216.

Thus, a simple actuation of the handle 244 and the camming pin 242 frictionally clamps the first and second support members 246, 248 within the first and second clamping members 212, 216, respectively, in selected rotational and axial positions. Conversely, the opposite movement of the handle 244 and rotation of the camming member 242 moves the head portion 228 of the tension bolt 222 relative to the first and second clamping members 212, 216 to reduce the forces that frictionally bind the first and second support members 246, 268 and thereby allow the first and second support members 246, 248 to be removed from the first and second clamping surfaces 214, 218, respectively.

The surgical clamp 210 enables a physician to quickly and easily adjust and re-adjust the rotational positions of the first and second clamping members 212, 216 as well as the position of the first and second support members 246, 248 within the first and second clamping members 212, 216 by providing a force perpendicular to an axis of the first and second surgical rods 246, 248. As a result, the surgeon can easily add an additional retractor where needed by disposing the clamp 210 about the second surgical rod 248 and providing a force perpendicular to an axis of the second support member 248 thereby disposing the second support member within the second clamping slot 217.

Further, the first support member 246, in this instance a retractor handle, is easily disposed within the first clamping slot 221 without having to thread an end of the first support member 246 through the first clamping slot 221. By rotating the handle 244 and camming pin 242 between the loosened position and the tightened position, the clamp 210 frictionally engages the first surgical rod 246 and the second surgical rod 248. As a result, adding an additional retractor within a surgical site is simple, quick and does not require the surgical team to break down any of the apparatus surrounding the surgical site.

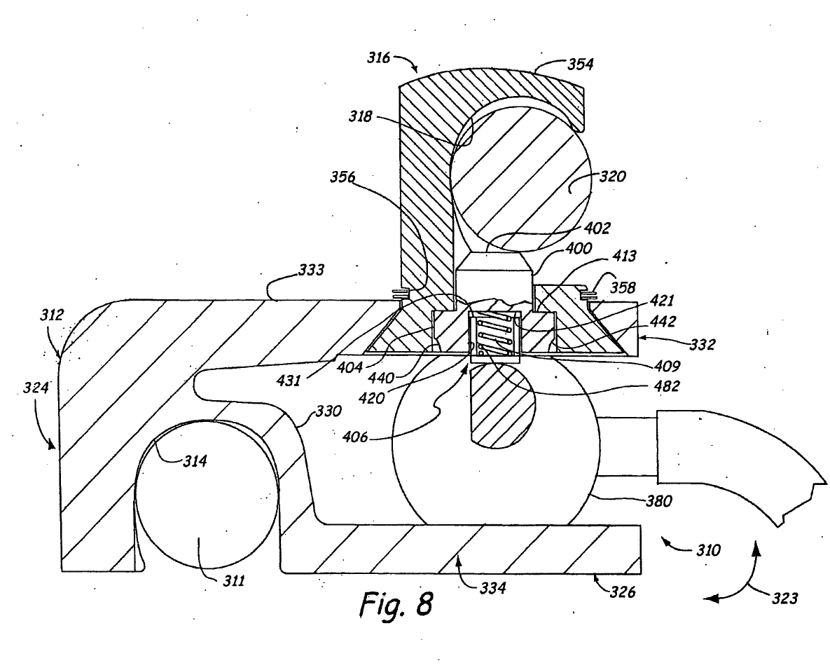

A second alternative embodiment of the surgical clamp of the present invention is generally illustrated in FIG. 8 at 310. The clamp 310 includes a first clamping member 312 having a first clamping surface 314 and a second clamping member 316 having a second clamping surface 318. Other components may be substituted for the second clamping member 316 such as, but not limited to, permanently attached retractors, or retractors that are attached using other types of clamps or fasteners.

To position the clamp 310 within the surgical site, a clamping end 324 of the first clamping member 312 is positioned proximate a retractor support arm 311. The first clamping surface 314 proximate the clamping end 324 is disposed about the portion of the retractor support arm 311 by applying manual force substantially perpendicular to an axis of the retractor support arm 311.

The first clamping member 312 also includes a fulcrum portion 328 proximate the clamping surface 314 and located between an upper leg portion 332 and a lower leg portion 334 as best illustrated in FIGS. 8 and 9. The fulcrum portion 328 allows the upper leg portion 332 to move with respect to the lower leg portion 334 so that the retractor support arm 311 may be clamped within the first clamping surface 314.

An actuating mechanism 380 is disposed within a recess 330 defined by the upper leg portion 332 and the lower leg portion 334 of the first clamping member 312 as illustrated in FIG. 8. The actuating mechanism 380 preferably is a camming pin.

The camming pin 380 is movable within the recess 330 and forces the upper and lower leg portions 332, 334 apart and causes the fulcrum portion 328 to flex. As the fulcrum portion 318 flexes, a clamping slot 338 defined by the first clamping surface 314 constricts such that the first clamping surface 314 frictionally engages the retractor support arm 311. The first clamping surface 314 is configured to generally conform to the cross-sectional configuration of the retractor support arm 311, but may be configured to conform to other shaped cross-sections.

Referring to FIGS. 8 and 9, the clamping slot 338 permits the first clamping member 312 to be transversely placed on the retractor support arm 311 without moving any other surgical equipment that has been previously disposed upon the retractor support arm 311. When the clamp 310 is in a loosened position, the first clamping member 312 is retained upon the retractor support arm 311 by a constricted entrance 339 to the clamping slot 338. A bead or raised surface 340 is disposed substantially along a length of the clamping slot 338 to constrict the entrance 339.

The constricted entrance 339 provides a preliminary clamping force around the portion of the retractor support arm 311 such that the constricted entrance 339 to the clamping slot 338 prevents the clamp 310 from slipping off the retractor support arm 311 when the clamp 310 is in a loosened position. The entrance 339 of the clamping slot 338 should not be so constricted to prevent use of manual force to position the first clamping member 312 about the portion of the retractor support arm 311. However, the entrance 339 of the clamping slot 338 should be sufficiently constricted to prevent the first clamping member 312 from accidentally slipping off of the retractor support arm 311.

The second clamping member 316 extends through a through bore 348 defined by a frustro-conical surface 349 within the upper leg portion 332 of the first clamping member 312. The second clamping member 316 includes a frustro-conical surface 350 at a proximal end 352 that cooperates with the frustro-conical surface 349 of the upper leg portion 332.

With the second clamping member 316 being positioned within the upper leg portion 332, an annular groove 356 on the second clamping member 316 is positioned above an upper surface 333 of the upper leg portion 332. A snap ring 358 is disposed within the annular groove 356 that rotatably captivates the second clamping member 316 within the upper leg portion 332 of the first clamping member 312 as illustrated in FIG. 8.

Referring to FIGS. 8 and 9, the second clamping surface 318 is positioned on a distal end portion 354 of the second clamping member 316. A retractor handle shaft 320 is disposed proximate an entrance 319 to an opening 317 defined by the second clamping surface 318. Manual force is applied to the retractor handle shaft 320 substantially perpendicularly to an axis to position the retractor handle shaft 320 within the opening 317 by displacing an end 402 of a slide pin 400 from the opening 317. The end 402 is positioned into the opening 317 through a through bore 372 extending through the second clamping member 316 along an axis 373 and intersects a bottom portion 361 of the clamping surface 318. With the end 402 displaced from the opening 317, the entrance 319 to the opening 317 is not constricted thereby allowing the retractor handle shaft 320 to be disposed within the opening 317.

With the retractor handle shaft 320 positioned within the opening 317, a compression spring 409, disposed between the slide pin 400 and a spacer 430 that contacts the camming pin 380, biases the end 402 of the slide pin 400 back into the opening 317. The end 402 and the arcuate upper portion 362 retains the retractor handle shaft 320 within the opening 317 such that the retractor handle shaft 320 is slidably positionable with respect to the second clamping surface 318.

The second clamping member 316 is positioned into a tightened position at approximately the same time that the camming pin 380 forces the first and second leg portions 332, 334 apart. When the second clamping member 316 is in the tightened position, the second clamping member 316 is non-rotatably fixed with respect to the first clamping member 312 and the retractor handle shaft 320 is frictionally engaged within the second clamping surface 318.

The camming pin 380 is positionable between a loosened position and a tightened position. In the loosened position, the second clamping member 316 is rotatable within the first clamping member 312. In the tightened position, the retractor support bar 311 is frictionally engaged within the first clamping surface 314 and a retractor support handle shaft 320 is frictionally engaged between the arcuate upper portion 362 of the second clamping surface 318 and the end 402 of the slide pin 400. Additionally, the second clamping member 316 is rotatably fixed with respect to the first clamping member 312 by a frictional engagement of the cooperating frustro-conical surfaces 349, 350.

The camming pin 380 is positioned within the recess 330 where the camming pin 380 includes a first cylindrical portion 388 and a second cylindrical portion 390 that are separated by the camming surface 382 which has an axis offset from the axis of rotation of the camming pin 380. Although a specific cam is described and illustrated in the drawings, other cam configurations are included within the present invention. By cam is meant an element having a raised surface or projecting part that when moved or rotated imparts an intermittent, alternate or variable motion.

The first and second cylindrical portions 388, 390 are rotatably positioned within first and second arcuate recesses 436, 438, respectively, within the lower leg portion 334 and first and second arcuate recesses 440, 442, respectively, within a lower portion 404 of the slide pin 400. The first arcuate recesses 436, 440 cooperate to rotatably retain the first cylindrical portion 388 and the second arcuate recesses 438, 442 cooperate to rotatably retain the second cylindrical portion 390 such that the camming pin 380 is rotatable between the loosened and tightened positions.

With the camming pin 380 in the loosened position, the spacer 430 is positioned on a flat surface 384 of the camming pin 380. With the spacer 430 positioned on the flat surface 384, the compression spring 409 biases a shoulder 413 towards the proximal end 350 of the second clamping member 316 and biases the end 402 of the slide pin 400 into the opening 317. A first end of the compression spring 409 is disposed within a cavity 420 of the slide pin 400. A second end of the compression spring 409 is disposed within the cavity 432 within the spacer 430 so the spacer 430 is slidably received within the cavity 420.

As the camming pin 380 is rotated into the tightened position, the spacer 430 is positioned on and raised by the camming surface 382. As the spacer 430 is raised, the compression spring 409 compresses until an end 431 of the spacer 430 contacts a surface 421 within the cavity 420. With the surfaces 421, 431 contacting and the camming pin 380 in the tightened position, the shoulder 413 is forced into the proximal end 352 of the second clamping member 316 creating a frictional engagement with the frustro-conical surfaces 349, 350. Additionally, the end 402 of the slide pin 400 is forced into the opening 317 such that the retractor handle shaft 320 is secured within the opening 317 by a frictional engagement between the end 402 of the slide pin 400, the retractor handle shaft 320 and the arcuate upper surface 362.

In operation, the camming pin 380 is positioned in the loosened position when the spacer 430 is positioned on the flat surface 384. With the camming pin 380 in the loosened position, the first clamping member 312 and the second clamping member 316 can accept the retractor support arm 311 and the retractor handle shaft 320, respectively, and the second clamping member 316 is rotatable with respect to the first clamping member 312.

The first clamping member 312 is disposed in a selected position on the retractor support arm 311 by positioning the constricted entrance 339 of the clamping slot 338 proximate the retractor support arm 311. Manual force is applied to the first clamping member 312 substantially perpendicular to an axis of the retractor support arm 311 to overcome the constricted entrance 339 of the clamping slot 338, thereby disposing the first clamping member 312 about the retractor support arm 311. With the camming pin 380 in the first position, the first clamping member 312 is slidably positionable on the retractor support arm 311.

With the first clamping member 312 positioned on the retractor support arm 311, the retractor handle shaft 320 is positioned proximate the constricted entrance 319 of the opening 317 within the second clamping member 316. Manual force is applied substantially perpendicular to an axis of the retractor handle shaft 320 to force the end 402 of the slide pin 400 from the opening 317 and position the retractor handle shaft 320 within the opening 317. With the retractor handle shaft 320 positioned within the opening 317, the compression spring 409 biases the end 402 of the slide pin 400 into the opening 317 such that the retractor handle shaft 320 is slidably retained within the second clamping surface 318.

To position the camming pin 380 into the tightened position, a handle 322 fixedly attached to the camming pin 380 is moved in the direction of arrows 323 as illustrated in FIG. 8, which rotates the camming pin 380 about the first and second cylindrical portions 388, 390. As the camming pin 380 is rotated about the first and second cylindrical portions 388, 390, the spacer 430 is raised in the cavity 420 and compresses the compression spring 409. A maximum force is placed upon the spacer 430 and slide pin 400 when the camming pin 380 is positioned into the tightened position when the end 431 of the spacer 430 contacts the surface 421 within the cavity 420 and applies the clamping force to the slide pin 400.

Referring to FIG. 8, with the camming pin 380 in the tightened position, the camming surface 382 pushes the spacer 430 upward to force the first and second leg portions 332, 334 apart. With the first and second leg portions 332, 334 forced apart, the fulcrum portion 328 flexes which causes the first clamping slot 338 to constrict such that the first clamping surface 314 frictionally engages the retractor support arm 311.

With the camming pin 380 in the tightened position, the shoulder 413 is forced into the proximal end 352 of the second clamping member 316 and creates a frictional engagement between the cooperating frustro-conical surface 349 of the through bore 348 in the upper leg portion 332 and the frustro-conical surface 350 of the second clamping member 316. The frictional engagement of the frustro-conical surfaces 349, 350 prevents rotational movement of the second clamping member 316 with respect to the first clamping member 312.

With the camming pin 380 in the tightened position, the first end 402 of the slide pin 400 is raised into the opening 417 through the through bore 372. The raised first end 402 of the slide pin 400 contacts the retractor handle shaft 320 and creates a frictional engagement between the arcuate upper portion 362 of the second clamping surface 318, the retractor handle shaft 320 and the first end 402 of the slide pin 400.

The retractor clamp 310 can be repositioned on the support arm 311 by first positioning the camming pin 380 into the loosened position such that the first clamping slot 338 is not constricted and detaching the first clamping member 312 from the retractor support arm 311. The first clamping member 312 is detachable from the retractor support arm 311 by applying manual force in an opposite direction as used to position the first clamping member 312 on the support arm 311. After the clamp 310 has been removed from the retractor support arm 311, the clamp 310 is repositionable on the retractor support arm 311 by positioning an entrance 339 to the clamping slot 338 against the retractor support arm 311 and applying manual force substantially perpendicular to the axis of that portion of the retractor support arm 311.

The retractor handle shaft 320 can also be repositioned within the second clamping member 316 without having to slide the retractor handle shaft 320 with respect to the second clamping surface 318. The retractor handle shaft 320 is removed from the second clamping member 316 by providing manual force in the opposite direction of the force used to position the retractor handle shaft 320 within the opening 317 of the second clamping member 316. The retractor handle shaft 320 can be reinserted into the opening 317 by reapplying manual force generally perpendicular to the axis of the retractor handle shaft 320.

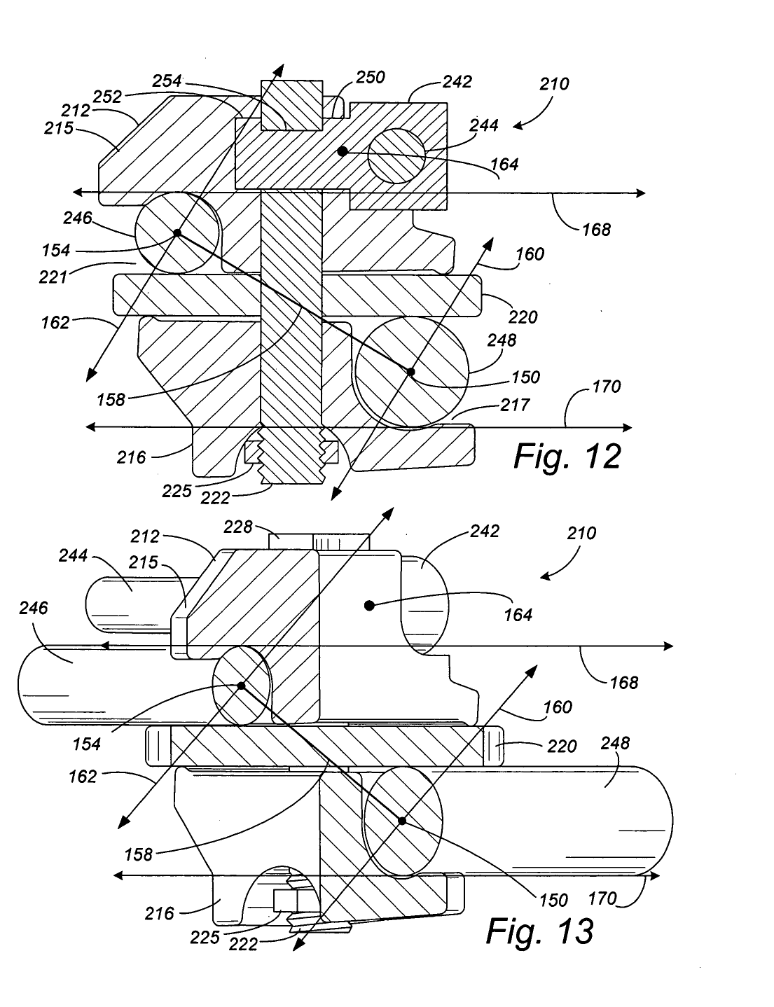

As will be appreciated throughout the preceding discussion, the location of the handle 22, 244, 322 for the clamp 10, 210, 310 relative to the retractor support arm 11, 248, 311 and relative to the retractor handle shaft 20, 246, 320 provides significant benefits during surgery. In particular, the location and “handle-in-between” way in which the handle 22, 244, 322 attaches to the body of the clamp 10, 210, 310 enables a very low profile, compact clamp, further understood with reference to the drawing of clamp 10 shown in FIGS. 10 and 11, the drawing of clamp 210 shown in FIGS. 12-14, and the drawing of clamp 310 shown in FIG. 15.

A support axis midpoint 150 is defined as the point where a midplane of the first clamping member 12, 216, 312 intersects the axis 152 (shown in FIG. 10) of the support frame arm 11, 248, 311, which is also the midpoint of the first clamping opening 38, 217, 314. A retractor shaft axis midpoint 154 is defined as the point where a midplane of the second clamping member 16, 212, 316 intersects the axis 156 (shown in FIG. 10) of the retractor handle shaft 20, 246, 320, which is also the midpoint of the axis defined by the opening 17, 221, 318. The “handle-in-between” nature of the clamps 10, 210, 310 is witnessed by the location that the handle 22, 244, 322 attaches to clamps 10, 210, 310 being at an intermediate location between the support axis midpoint 150 and retractor shaft axis midpoint 154.

More exactly, a midpoint connection line 158 can be drawn between the support axis midpoint 150 and the retractor shaft axis midpoint 154. A support profile plane 160 is defined as the plane through the support axis midpoint 150 which is perpendicular to the midpoint connection line 158. A retractor profile plane 162 is defined as the plane through the retractor shaft axis midpoint 154 which is perpendicular to the midpoint connection line 158. In each case of FIGS. 10-13 and 15, the point 164 that the handle 22, 244, 322 attaches to clamps 10, 210, 310 is between the support profile plane 160 and the retractor profile plane 162.

The point 164 that the handle attaches to the clamp may coincide with the end of a component of the handle (as in FIGS. 10 and 11). Alternatively, the point 164 that the handle attaches to the clamp may be interior to a component of the handle (as in FIGS. 12-15). Either way, the point 164 that the handle 22, 244, 322 attaches to the clamp 10, 210, 310 is defined by the profile of the clamp body which does not move with the handle relative to the structure that does move with the handle. Forming the handle in multiple components which partially perform other functions, such as wedge 80 providing part of the handle by extending beyond the profile defined by the upper and lower legs 32, 34, does not affect the location of the point 164 that the handle attaches to the clamp.

The “handle-in-between” and low profile nature of the clamps 10, 210, 310 is important when assessing the likelihood that the clamp 10, 210, 310 or other part of the surgical retractor structure will interfere with the surgeon's access to the surgical site, and assessing the likelihood that the handle 22, 244, 322 could get in the way or be inadvertently contacted during the surgery. In each embodiment, with the handle 22, 244, 322 attached at a location 164 between the support profile plane 160 and the retractor profile plane 162, but with a single handle operating both clamping members, the clamp 10, 210, 310 can be positioned onto the support frame arm 11, 248, 311 with minimal “underclearance”. For instance, in the first and third embodiment, the clamp 10, 310 can be attached to the support frame arm 11, 311 at a location where the support frame arm 11, 311 is positioned above and contacting the patient's body or other underlying structure 166 (shown conceptually in FIG. 11). The surgeon can position the support frame arm 11, 311 in the lowest possible position without affecting the operation of the clamp 10, 310. In the second embodiment, enough room must be left underneath the support frame arm 248 for the first end 219 of the clamping member 216 to extend between the support frame arm 248 and the underlying patient's body or other underlying structure. A complete throw of the handle 22, 244, 322 is possible even in such a close-in arrangement.

In each embodiment, with the handle 22, 244, 322 attached at a location between the support profile plane 160 and the retractor profile plane 162, but with a single handle operating both clamping members, the retractor handle shaft 20, 246, 320 can be attached to the clamp 10, 210, 310 with minimal “overclearance”. In the first and third embodiment, the only portion of the clamp 10, 310 which extends in a sight line over the retractor handle shaft 20, 320 is the small height of the distal end portion 54, 354 of the second clamping member 16, 316. The complete throw of the handle 22, 322 does not place the handle in a position to interfere with sight lines into the surgical arena, and, most importantly, the tightened position of the handle 22, 322 does not interfere with sight lines into the surgical arena. In the second embodiment, the only portion of the clamp 210 which extends in a sight line over the retractor handle shaft 246 is the first end 215 of the first clamping member 212. Even though the throw of the handle 244 takes it over the top into a sight line into the surgical arena, neither the loosened position nor (more importantly) the tightened position of the handle 244 interferes with sight lines into the surgical arena. In all these embodiments, with the handle 22, 244, 322 attached at a location between the support profile plane 160 and the retractor profile plane 162, the handle 22, 244, 322 is unlikely to be inadvertently contacted during the surgery.

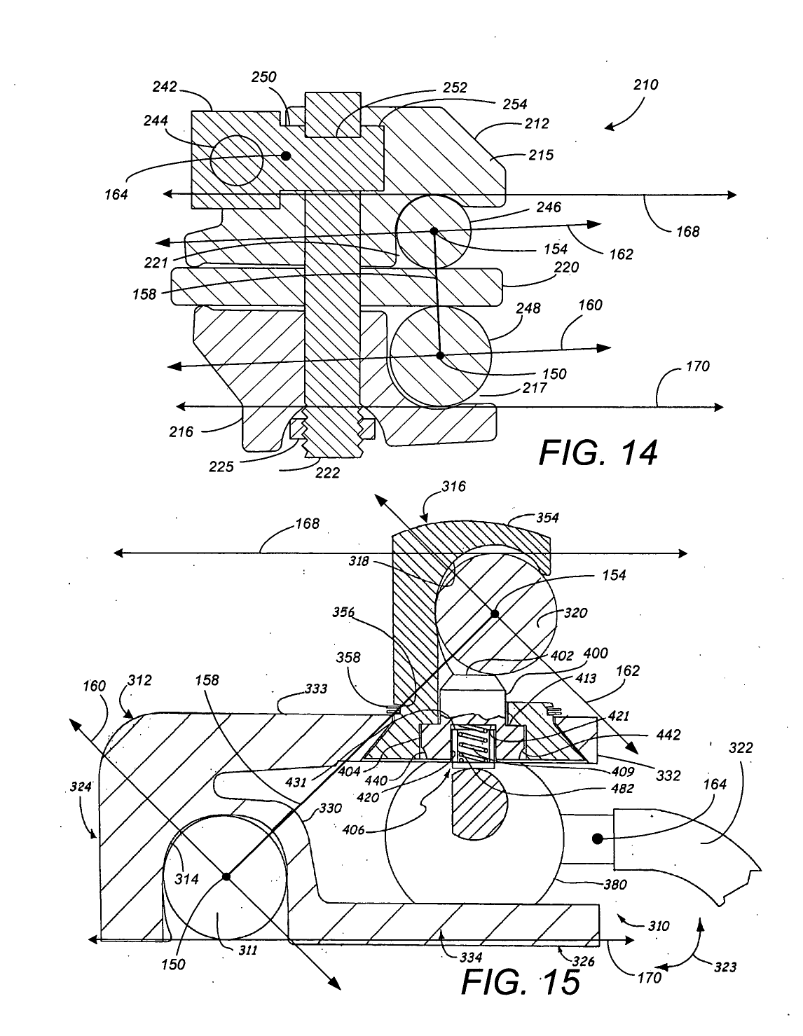

For the first embodiment and the third embodiment, pivoting the second clamping member 16, 316 relative to the first clamping member 12, 312 does not significantly change the position of the retractor shaft axis midpoint 154 relative to the support axis midpoint 150. All orientations of the clamp 10, 310 are “handle-in-between”, and all orientations of the clamp 10, 310 place the handle 22, 322 and the second clamping member 16, 316 on the same side of the support profile plane 160. In contrast, the second embodiment is a structure wherein pivoting of the second clamping member 216 relative to the first clamping member 212 does significantly change the position of the retractor shaft axis midpoint 154 relative to the support axis midpoint 150. To show this, FIG. 12 maps the retractor shaft axis midpoint 154 relative to the support axis midpoint 150 with the clamp 210 in the position of FIGS. 6 and 7, FIG. 13 maps the retractor shaft axis midpoint 154 relative to the support axis midpoint 150 with the first clamp member 212 pivoted 90° relative to the position of FIGS. 6 and 7, and FIG. 14 maps the retractor shaft axis midpoint 154 relative to the support axis midpoint 150 with the first clamp member 212 pivoted 180° relative to the position of FIGS. 6 and 7. Of these three positions, the mapping of FIG. 13 is most important, because most uses of the clamp 210 involve the retractor handle shaft 246 extending perpendicular or nearly perpendicular to the support frame arm 248. However, as shown in FIG. 14, the clamp 210 can be turned to a position wherein the handle 244 attaches at a location 164 which is above the retractor profile plane 162, i.e., attached at a location 164 which is (due to pivoting of the two clamping members 212, 216) no longer “in-between” the support profile plane 160 and the retractor profile plane 162. Thus, the second embodiment has orientations which are not “handle-in-between”, whereas all orientations of the clamps 10, 310 of the first and third embodiments are “handle-in-between” with the handle 22, 322 attaching between the support profile plane 160 and the retractor profile plane 162. The second embodiment could be modified to be “handle-in-between” in all orientations by lowering the height of the handle 244 and/or raising the height of the retractor handle shaft 246 relative to the washer 220.

With both the retractor handle shaft 20, 246, 320 and the support frame arm 11, 248, 311 extending horizontally, the handle 22, 244, 322 should be no higher than that of FIGS. 12-14 to provide a “low profile” to the device. In this horizontal orientation, there is essentially no height separation between the bottom of the handle 244 and the top plane 168 defined by pivoting of the retractor handle shaft 246. The retractor handle shaft 20, 246, 320 can be pivoted to a plurality of orientations defining a horizontal shaft top plane 168, which extends above and does not intersect the horizontal axis of the support frame arm 11, 248, 311. A support frame arm bottom plane 170 can be defined as parallel to the shaft top plane 168 and containing the bottom of the support frame arm 11, 248, 311. While not “handle-in-between” in all orientations, the embodiment of FIGS. 12-14 benefits by having the retractor shaft 246 very close in height to the height of the support frame arm 248, separated by only the height of the washer 220. The handle 22, 322 of the first and third embodiments is at an even better height, above the support frame arm bottom plane 170 and below the shaft top plane 168. Similar to the “handle-in-between” nature of the clamps 10, 210, 310, this low height of the handle 22, 244, 322, while still allowing adequate access to the handle 22, 244, 322, allows minimal interference with sight lines into the surgical arena and minimum chance of the handle 22, 244, 322 interfering with the surgical procedure or being inadvertently contacted during surgery.

In the first embodiment, the handle throw plane always contains or is substantially parallel to the axis of the retractor support arm 11, enabling tightening of the handle 22 to be achieved with a scissors action between the handle 22 and the retractor support arm 11. In the second embodiment, the handle throw plane is always substantially parallel to the retractor handle shaft 246. Whether the handle 244 can be tightened by a scissors action between the handle 244 and the retractor handle shaft 246 depends upon the lateral separation between the handle throw plane and the retractor handle shaft 246. If desired to minimize the lateral separation between the handle throw plane and the retractor handle shaft 246, the handle 244 and camming pin 242 may be reversed relative to the tension bolt 222, such that the handle 244 and the retractor handle shaft 246 reside on the same side of the tension bolt 222 rather than having the tension bolt 222 between the handle 244 and the retractor handle shaft 246.

In the third embodiment, the handle throw plane is always perpendicular to the support arm 311, and will only be parallel to the retractor handle shaft 320 when the retractor handle shaft 320 is perpendicular to the support arm 311. Because the handle throw plane is not always parallel to (or containing) either the support arm 311 or the retractor handle shaft 320, the third embodiment does not always permit a scissors-type tightening action.

Yet another advantage of the first and third embodiments can be seen by comparing the pivot axis 73, 373 for the second clamp member 16, 316 relative to the axis of the retractor handle shaft 20, 320, and further relative to the pivot axis for the handle 22, 322. That is, in the first and third embodiments, the pivot axis 73, 373 for the second clamp member 16, 316 substantially intersects the axis 156 of the retractor handle shaft 20, 320. By having these two axes intersect, the clamp 10 provides a very balanced look and feel, and further minimizes the likelihood of damage due to over-torqueing of components of the clamp 10. After the clamp 10, 310 has been loosely attached onto the retractor support arm 11, 311, movement of the retractor handle shaft 20, 320 occurs entirely by pivoting, rather than movement in a sweeping arc.

By further having the pivot axis for the handle 22, 322 substantially intersect or substantially coincide with the pivot axis 73, 373 for the second clamp member 16, 316, users intuitively understand the proper direction to apply force to the handle 22, 322 to tighten the clamp 10, 310 without providing unnecessary forces which might dislodge the clamp 10, 310 from the support arm 11, 311 or from the retractor shaft 20, 320. After the clamp 10, 310 has been loosely attached onto the retractor support arm 11, 311, the tightening movement of the handle 22, 322 is substantially entirely a pivoting movement about an identified location substantially intersecting or substantially coinciding with the pivot axis 73, 373.

Thus it can be seen that the location and orientation of the handle 22, 244, 322 relative to the clamp 10, 210, 310, and particularly the “handle-in-between” and low height of the handle 22, 244, 322 and the scissors-tightening of the handle 22, 244, provide many advantages during the surgical procedure which are not provided by prior art clamps. While many linkage mechanisms can be used to translate tightening and loosening forces between the handle 22, 244, 322 and the clamp 10, 210, 310, the preferred linkages cost effectively and efficiently provide the “handle-in-between”, low height and scissors-tightening advantages.

Although the present invention has been described with reference to preferred embodiments, workers skilled in the art will recognize that changes may be made in form and detail without departing from the spirit and scope of the invention. For instance, while the preferred embodiment includes two clamping members, certain aspects of the invention could be practiced with a single clamping member, such as integral with and/or permanently attached to either the support frame arm 11, 248, 311 or the retractor shaft 20, 246, 320. While particular linkages are described which enable the handle 22, 244, 322 to simultaneously control both clamping members, it is recognized that many other types of linkages could be used while still obtaining the handle orientation benefits of the present invention.

1. A scissors clamp comprising:
a support frame attachment structure for attaching the scissors clamp to a support frame arm, the support frame arm defining a support frame arm axis;
a clamp member having an opening for receiving a rod, the opening changing dimensions to receive the rod in a loosened clamp position and in a tightened clamp position, the opening defining a rod axis, the clamp member when in a loosened clamp position being pivotally connected to the support frame attachment structure, such that movement of the pivotal connection causes the rod axis to define a rod movement travel path; and
a handle pivotable in a handle throw plane, pivoting of the handle moving the clamp member between its loosened clamp position and its tightened clamp position, wherein the handle throw plane always substantially contains or is parallel to one of the support frame arm axis and the rod axis regardless of movement of the pivotal connection through the rod movement travel path.

2. The scissors clamp of claim 1, wherein the support frame attachment structure comprises a second clamp member having an opening for receiving a support frame rod, the second clamp member having a loosened second clamp position and changing dimensions to have a tightened second clamp position.

3. The scissors clamp of claim 2, wherein pivoting of the handle moves the second clamp member between its loosened second clamp position and its tightened second clamp position.

4. The scissors clamp of claim 1, wherein the handle throw plane always substantially contains or is parallel to the support frame arm axis, and wherein the clamp member and the handle are both on the same side of the support frame arm.

5. The scissors clamp of claim 1, wherein the handle throw plane is substantially parallel to or contains the rod movement travel path.

6. The scissors clamp of claim 1, wherein the handle throw plane is disposed at an angle to the rod movement travel path.

7. The scissors clamp of claim 1, wherein the handle throw plane always substantially contains or is parallel to the rod axis.

8. The scissors clamp of claim 1, wherein the handle has a longitudinal handle axis, the handle orientation relative to the rod axis being maintained by the scissors clamp such that, when the handle tightens to the tightened clamp position, the longitudinal handle axis is generally parallel to the rod axis.

9. The scissors clamp of claim 1, wherein the handle has a longitudinal handle axis, the handle orientation relative to the support frame arm axis being maintained by the scissors clamp such that, when the handle tightens to the tightened clamp position, the longitudinal handle axis is generally parallel to the support frame arm axis.

10. A handle-in-between surgical clamp comprising:
a first clamp member having a first opening for receiving a first rod, the first opening having changeable dimensions to receive the first rod in a loosened first clamp position and in a tightened first clamp position, the first opening defining a first rod axis;
a second clamp member having a second opening for receiving a second rod, the second opening having changeable dimensions to receive the second rod in a loosened second clamp position and in a tightened second clamp position, the second clamp member attached to the first clamp member; and
a handle for changing the dimensions of at least one of the first opening and the second opening, the handle attached at a location between the first clamp member and the second clamp member.

11. The surgical clamp of claim 10, wherein, with the handle in a loosened position the second clamp member is pivotable relative to the first clamp member, and wherein the location that the handle attaches is between the first clamp member and the second clamp member regardless of pivoting orientation of the second clamp member relative to the first clamp member.

12. The surgical clamp of claim 10, wherein the handle pivots through a handle throw plane to simultaneous change the first clamp member and the second clamp member between loosened positions and tightened positions.

13. The surgical clamp of claim 12, wherein, with the handle in a loosened position the second clamp member is pivotable relative to the first clamp member, and wherein pivoting of the handle to a tightened position simulataneously tightens the attachment of the second clamp member to the first clamp member to prevent pivoting.

14. A low profile surgical clamp comprising:
a first clamp member having a first opening for receiving a first rod, the first opening defining a first rod axis and a first rod bottom line;
a second clamp member having a second opening for receiving a second rod, the second opening defining a second rod axis and a second rod top line, the second clamp being pivotally attached to the first clamp member such that the second rod top line can be positioned at a plurality of orientations within a second rod top plane which does not intersect the first rod axis, with a first rod bottom plane being defined as parallel to the second rod top plane and containing the first rod bottom line; and
a handle for securing the first opening against pivoting relative to the second opening, the handle being positionable substantially at or within the space between the first rod bottom plane and the second rod top plane.

15. The low profile surgical clamp of claim 14, wherein the handle is positionable at a tightened position such that the handle is entirely within the space between the first rod bottom plane and the second rod top plane.

16. A surgical clamp comprising:
a first clamp member having a first opening for receiving a first rod, the first opening changing dimensions to receive the first rod in a loosened first clamp position and in a tightened first clamp position, the first opening defining a first rod axis;
a second clamp member having a second opening for receiving a second rod, the second opening changing dimensions to receive the second rod in a loosened second clamp position and in a tightened second clamp position, the second opening defining a second rod axis, the second clamp being pivotally attached to the first clamp member such that the second rod axis can be positioned at a plurality of orientations wherein the second rod axis does not intersect the first rod axis, pivoting of the second rod axis occurring about a rod pivot axis, wherein the rod pivot axis intersects at least one of the first rod axis and the second rod axis; and
a handle moving the first clamp between the loosened first clamp position and the tightened first clamp position, the handle extending longitudinally and pivoting to provide a mechanical advantage in tightening force applied.

17. The surgical clamp of claim 16, wherein the handle pivots about a handle pivot axis in a handle plane, wherein the handle plane contains or is substantially parallel to the rod pivot axis.

18. The surgical clamp of claim 17, wherein the handle pivot axis intersects the rod pivot axis.

19. A surgical clamp comprising:
a first clamp member having a rod opening for receiving a first rod, the first clamp member having a pin bore intersecting the rod opening;
a pin disposed for longitudinal sliding in the pin bore such that an end of the first pin can bias the first rod within the first clamp member to tighten the first rod within the first clamp member;
a handle for sliding the first pin longitudinally within the pin bore; and
a second clamp member having a second opening for receiving a second rod, the second clamp member being attached to the first clamp member.

20. The surgical clamp of claim 19, wherein the second clamp member is pivotally attached to the first clamp member such that the second rod axis can be positioned at a plurality of orientations wherein the second rod axis does not intersect the first rod axis.

21. A surgical clamp comprising:
a first clamp member having a first opening for receiving a first rod, the first opening changing dimensions to receive the first rod in a loosened first clamp position and in a tightened first clamp position;
a second clamp member having a second opening for receiving a second rod, the second opening changing dimensions to receive the second rod in a loosened second clamp position and in a tightened second clamp position, the second clamp being pivotally attached to the first clamp member such that the second rod axis can be positioned at a plurality of orientations, the pivotal attachment including mating frustro-conical surfaces about a clamp pivot axis;
a wedge which, upon pivoting rotation, increases in an operative dimension to bias the mating frustro-conical surfaces to frictionally engage each other and thereby tighten the second clamp member against further pivoting relative to the first clamp member; and
a handle coupled to the wedge to pivotally rotate the wedge.

22. The surgical clamp of claim 21, wherein the wedge is a cam.

23. A surgical clamping system comprising:
a support frame, at least a connection portion of the support frame defining a support frame axis;
a shaft, at least a connection portion of the shaft defining an axis extending over the support frame axis;
a clamp member for connecting the connection portion of the shaft to the connection portion of the support frame, the clamp member having a tightened position wherein the shaft is frictionally fixed relative to the support frame and a loosened position wherein the shaft is moveable relative to the support frame; and
a handle for moving the clamp member between the loosened position and the tightened position, which, in the tightened position, resides beneath the shaft axis and on a same side of the support frame axis as the connection portion of the shaft.
